(19) United States
(12) Patent Application Publication
(10) Pub. No.: US 2020/0170647 A1
(43) Pub. Date: Jun. 4, 2020

(54) VASO-OCCLUSIVE DEVICE

(71) Applicants: STRYKER CORPORATION, Kalamazoo, MI (US); STRYKER EUROPEAN HOLDINGS I, LLC, Kalamazoo, MI (US)

(72) Inventors: Hancun CHEN, San Ramon, CA (US); Andrew S. LEE, San Jose, CA (US)

(21) Appl. No.: 16/208,860

(22) Filed: Dec. 4, 2018

(51) Int. Cl. A61B 17/12; A61F 2/07

(52) U.S. Cl. CPC A61B 17/12113; A61B 17/12145; A61F 2/958; A61B 17/1215; A61F 2/07

(57) ABSTRACT

A vaso-occlusive device comprises an elongate vaso-occlusive structure configured for implantation in an aneurysm sac. The vaso-occlusive structure has a delivery configuration when restrained within a delivery catheter and has a deployed configuration when released from the delivery catheter into the aneurysmal sac. At least a portion of the vaso-occlusive device is composed of a gold-platinum (AuPt) alloy.

### FIELD

The present disclosure relates generally to medical devices and intravascular medical procedures and, more particularly, to devices and methods for occluding vascular defects, such as aneurysms.

### BACKGROUND

Vaso-occlusive devices or implants are used for a wide variety of reasons, including treatment of intra-vascular aneurysms. An aneurysm is a dilation of a vessel, such as a blood vessel, that may pose a risk to a patient's health due to rupture, clotting, or dissection. For example, rupture of an aneurysm in a patient's brain may cause a stroke, and lead to brain damage and death. Cerebral aneurysms may be detected in a patient, e.g., following seizure or hemorrhage, and may be treated by applying vaso-occlusive devices.

Commonly used vaso-occlusive devices include soft, helically wound coils formed by winding a platinum (or platinum alloy) wire strand about a “primary” mandrel.

The coil is then wrapped around a larger, “secondary” mandrel, and heat treated to impart a secondary shape. For example, U.S. Pat. No. 4,994,069, issued to Ritchart et al., which is fully incorporated herein by reference as though set forth in full, describes a vaso-occlusive device that assumes a linear, helical primary shape when stretched for placement through the lumen of a delivery catheter, and a folded, convoluted secondary shape when released from the delivery catheter and deposited in the vasculature. In order to better frame and fill aneurysms, complex three-dimensional secondary shapes can be imparted on vaso-occlusive devices and the stiffness/flexibility of vaso-occlusive devices can be modified.

In order to deliver the vaso-occlusive devices to a desired site in the vasculature, e.g., within an aneurysmal sac, it is well-known to first position a small profile delivery catheter or “micro-catheter” at the site using a guidewire. Typically, the distal end of the micro-catheter is provided, either by the attending physician or by the manufacturer, with a selected pre-shaped bend, e.g., 45°, 26°, “J”, “S”, or other bending shape, depending on the particular anatomy of the patient, so that it will stay in a desired position for releasing one or more vaso-occlusive device(s) into the aneurysmal sac once the guidewire is withdrawn. A delivery or “pusher” assembly or “wire” is then passed through the micro-catheter until a vaso-occlusive device coupled to a distal end of the delivery assembly is extended out of the distal end opening of the micro-catheter and into the aneurysmal sac. Once in the aneurysmal sac, portions of the vaso-occlusive device may deform or bend to allow more efficient and complete packing.

The vaso-occlusive device is then released or “detached” from the distal end of the delivery assembly, and the delivery assembly is withdrawn back through the micro-catheter. Depending on the particular needs of the patient, one or more additional vaso-occlusive devices may be pushed through the micro-catheter and released into the same aneurysmal sac.

Significantly, fluoroscopy is typically used to visualize vaso-occlusive devices during delivery into an aneurysm, while magnetic resonance imaging (MRI) is typically used to visualize the treatment site post-procedure (e.g., a few weeks after initial treatment of the aneurysm) to ensure that the aneurysmal sac is properly occluded. As such, it is important that vaso-occlusive devices be constructed in a manner that enables their radiopacity during treatment of the aneurysm, while minimizing any visualization obscuring artifacts created during the post-procedure MRI (i.e., being MRI-compatible). It is also paramount that such vaso-occlusive devices be “soft” (i.e., be laterally flexible or conformable), and thus atraumatic, to prevent rupturing of the delicate tissues of the aneurysm.

It is also important that such vaso-occlusive devices be chronically retained within the aneurysm. However, aneurysms with larger mouths, commonly known as “wide neck aneurysms,” present difficulty in the placement and retention of vaso-occlusive devices within the aneurysm sacs, particularly with small and relatively thin vaso-occlusive coils which lack the substantial secondary shape strength to maintain in position within such aneurysm sacs no matter how skillfully they are placed. For this reason, a stent or a balloon must be deployed in the vessel adjacent the neck region of the aneurysm to ensure that the vaso-occlusive coils are retained within the aneurysmal sac, thereby complicating the procedure. To address this particular issue, vaso-occlusive devices at least partially composed of a braided (or woven) structure have been developed. Such braided vaso-occlusive devices provide more coverage and a more effective backbone across the necks of aneurysms, and can thus be effectively retained within wide neck aneurysms without the need to deploy supplemental aneurysm-retaining devices, such as balloon or stents.

However, regardless of whether coiled or braided vaso-occlusive devices are used, conventional vaso-occlusive device delivery systems require that that such vaso-occlusive devices be relatively short and limited in expandability, otherwise they are difficult (if not impossible) to push and/or retrieve to/from the microcatheter. Unfortunately, small (short) vaso-occlusive devices are less desirable, since delivery of such small vaso-occlusive devices into an aneurysmal sac may require a longer and more involved procedure. For example, a 7 mm diameter neurological aneurysmal sac may typically be filled with five to seven individual spring shaped coils, resulting in a longer and more complicated procedure than if the number of devices was reduced.

Theoretically, the lengths of vaso-occlusive devices may be increased to reduce the number of such vaso-occlusive devices needed to treat an aneurysm. However, increasing the length of a vaso-occlusive device necessarily increases the friction of such vaso-occlusive device and the lumen of the delivery catheter. As such, the columnar strength of such vaso-occlusive device must be increased (e.g., by selecting a material with a high Young's modulus or increasing the diameter of the wire from which the vaso-occlusive device is formed) and/or the diameter of the delivery catheter must be increased to ensure that the vaso-occlusive device can be delivered into the aneurysm. However, as discussed above, it is important that both the diameter of the delivery catheter be as small as possible to allow the aneurysm to be accessed through a very small vasculature, and the vaso-occlusive device be soft enough to prevent trauma to the delicate tissues of the aneurysm.

Materials that enable a relatively long vaso-occlusive to have the necessary columnar strength to be delivered through a relatively small diameter delivery catheter, while satisfying the other countervailing requirements, including softness, radiopacity, and MRI-compatibility requirements, are very limited.

For example, known materials having a relatively high Young's modulus and relatively high radiopacity, such as platinum-tungsten (PtW) alloy from which vaso-occlusive coils are typically manufactured, can be used in an attempt to provide the necessary columnar strength for a relatively long vaso-occlusive device; however, the diameter of the wires from which such vaso-occlusive device is manufactured must be reduced to satisfy the softness requirements while allowing the vaso-occlusive device to fit within a small diameter delivery catheter. As a result, the vaso-occlusive device would have a degraded radiopacity and a decreased columnar strength that would require a shortened vaso-occlusive device and/or larger diameter delivery catheter.

As another example, known materials having a relatively low Young's modulus and low radiopacity, such as nitinol, can be used in an attempt to provide the necessary softness for a vaso-occlusive device; however, such vaso-occlusive device would not have a desirable radiopacity and columnar strength necessary to increase the length of the vaso-occlusive device. Furthermore, the heating process used to set nitinol in its pre-determined shape results in a surface oxide that may crack and release a toxic nickel. Thus, such oxide must be removed from the vaso-occlusive device using a costly and time-consuming process.

As still another example, known materials having a relatively medial Young's modulus and low radiopacity, such as titanium and the like, can be used in an attempt to provide the necessary columnar strength for a relatively long and soft vaso-occlusive device if an optimum diameter is selected for the wire from which such vaso-occlusive device is manufactured; however, such vaso-occlusive device would not exhibit the necessary radiopacity.

There, thus, is an ongoing need to provide a vaso-occlusive device that satisfies the foregoing requirements.

### SUMMARY

In accordance with one aspect of the present inventions, a vaso-occlusive device comprise an elongate vaso-occlusive structure (e.g., at least 5 cm in length) configured for implantation in an aneurysm sac. The vaso-occlusive structure has a delivery configuration when restrained within a delivery catheter and has a deployed configuration when released from the delivery catheter into the aneurysmal sac. At least portion of the vaso-occlusive structure is composed of a gold-platinum (AuPt) alloy, e.g., comprising platinum within the range 25%-40% by weight, and having a Young's modulus less than 25×10⁶ pounds per square inch (psi). The vaso-occlusive structure may be further composed of one or both of Iridium and Tungsten.

In one embodiment, the vaso-occlusive structure comprises a mesh portion (e.g., a braid portion) composed of the AuPt alloy. The mesh portion may have a bending stiffness less than 150 mN/mm. The entirety of the vaso-occlusive structure comprises the mesh portion, or alternatively, the vaso-occlusive structure may further comprise two helically wound coil portions disposed at opposite ends of the mesh portion. The coil portions may be composed of the AuPt alloy. The mesh portion may comprise at least one wire (e.g., 8-96 wires), each having, e.g., a minimum cross-sectional dimension in the range of 0.0008″-0.004″. Each of the wire(s) may, e.g., have a circular cross-section or a rectangular cross-section. Each wire may have, e.g., a single strand or a twisted strand. If the mesh is a braid portion, the braid portion may have an unconstrained braid angle in the range of 20°-130°, preferably in the range of 20°-60°. The mesh portion may have an expanded geometry, e.g., having a circular cross-section or a flat cross-section (e.g., having a width in the range of 0.5 mm-5.0 mm, preferably in the range of 1.0 mm-2.0 mm).

The vaso-occlusive device may be incorporated into a vaso-occlusive further comprising a pusher member to which the vaso-occlusive device is detachably coupled (e.g., electrolytically). The vaso-occlusive assembly may be incorporated into a vaso-occlusive treatment system that comprises the delivery catheter in which the vaso-occlusive assembly is disposed.

Other and further aspects and features of embodiments of the disclosed inventions will become apparent from the ensuing detailed description in view of the accompanying figures.

### BRIEF DESCRIPTION OF THE DRAWINGS

The drawings illustrate the design and utility of preferred embodiments of the present invention, in which similar elements are referred to by common reference numerals. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention, which is defined only by the appended claims and their equivalents. In addition, an illustrated embodiment of the disclosed inventions needs not have all the aspects or advantages shown. An aspect or an advantage described in conjunction with a particular embodiment of the disclosed inventions is not necessarily limited to that embodiment and can be practiced in any other embodiments even if not so illustrated. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a side view of an vaso-occlusive treatment system constructed according to one embodiment of the present inventions, particularly showing the vaso-occlusive within the delivery catheter in a delivery configuration;

FIG. 2 is a side view of the vaso-occlusive treatment system of FIG. 1, particularly showing the vaso-occlusive device deployed from the delivery catheter in an expanded configuration;

FIG. 3 is a plan view of a vaso-occlusive structure of the vaso-occlusive treatment system of FIG. 1, deployed within an aneurysmal sac;

FIG. 4 is a plan view of a mesh portion of the vaso-occlusive structure of the vaso-occlusive treatment system of FIG. 1;

FIG. 5A is a cross-sectional view of one embodiment of a wire used in the mesh portion of FIG. 4;

FIG. 5B is a cross-sectional view of another embodiment of a wire used in the mesh portion of FIG. 4;

FIG. 5C is a cross-sectional view of still another embodiment of a wire used in the mesh portion of FIG. 4;

FIG. 6A is a cross-sectional view of one embodiment of the mesh portion of the vaso-occlusive treatment system of FIG. 1;

FIG. 6B is a cross-sectional view of another embodiment of the mesh portion of the vaso-occlusive treatment system of FIG. 1;

FIG. 7A is a fluoroscopic image of a prototype of a vaso-occlusive structure constructed in accordance with the present inventions;

FIG. 7B is a fluoroscopic image of a prototype of another vaso-occlusive structure constructed in accordance with the present inventions;

FIG. 8A is an MRI image of a prototype of an aneurysm filled with conventional Pt/8W vaso-occlusive coils;

FIG. 8B is an MRI image of a prototype of an aneurysm filled with Au/Pt vaso-occlusive coils constructed in accordance with one embodiment of the present inventions;

FIG. 9 is a side view of an vaso-occlusive treatment system constructed according to another embodiment of the present inventions, particularly showing the vaso-occlusive within the delivery catheter in a delivery configuration; and

FIG. 10 is a side view of the vaso-occlusive treatment system of FIG. 9, particularly showing the vaso-occlusive device deployed from the delivery catheter in an expanded configuration.

### DETAILED DESCRIPTION OF THE ILLUSTRATED EMBODIMENTS

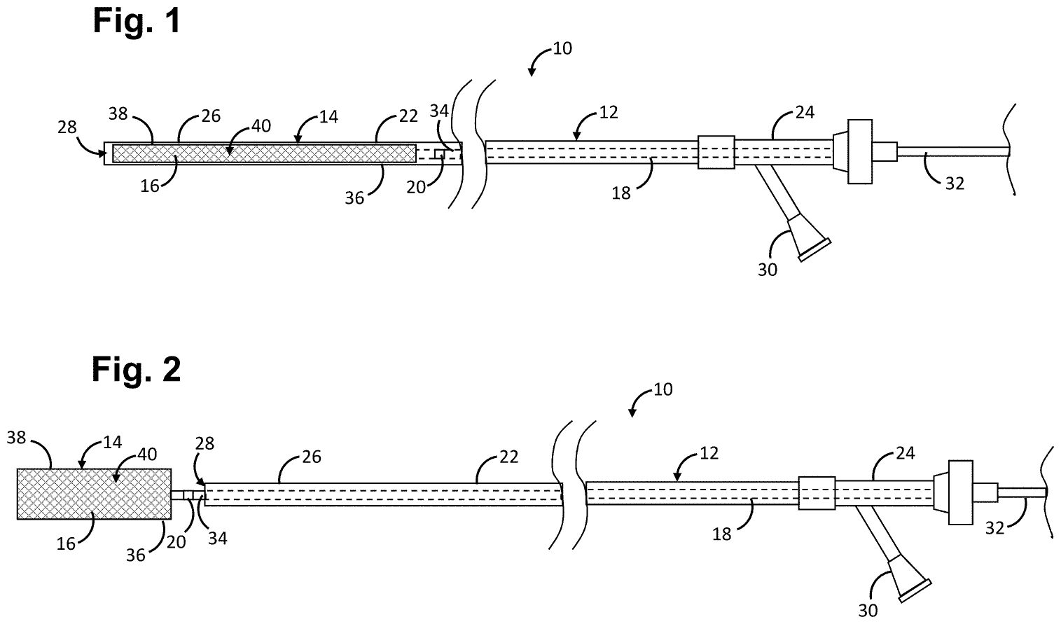

Referring to FIGS. 1 and 2, one embodiment of a vaso-occlusive treatment system 10 constructed in accordance with the present inventions will now be described. The vaso-occlusive treatment system 10 comprises a delivery catheter 12 and a vaso-occlusive assembly 14 slidably disposed within the delivery catheter 12. The vaso-occlusive assembly 14 comprises a vaso-occlusive structure 16 and a pusher member 18 to which the vaso-occlusive structure 16 is detachably coupled at a junction 20.

The delivery catheter 12 has a tubular configuration, and can, e.g., take the form of a micro-catheter or the like. The delivery catheter 12 comprises an elongate sheath body 22 having a proximal portion 24 and a distal portion 26, and a lumen 28 (shown in phantom) extending through the sheath body 22 between the proximal portion 24 and the distal portion 26. The proximal portion 24 of the sheath body 22 remains outside of the patient and accessible to the operator when the vaso-occlusive treatment system 10 is in use, while the distal portion 26 of the sheath body 22 is sized and dimensioned to reach remote locations of a vasculature and is configured to deliver the vaso-occlusive structure 16 to an aneurysm. The delivery catheter 12 may have at least one port 30 in fluid communication with the lumen 28 of the delivery catheter 12, which is used to introduce fluids into the sheath body 22. The vaso-occlusive assembly 14 is disposed in the lumen 28 of the delivery catheter 12, as better appreciated in FIG. 1.

The delivery catheter 12 may include one or more, or a plurality of regions along its length having different configurations and/or characteristics. For example, the distal portion 26 of the sheath body 22 may have an outer diameter less than the outer diameter of the proximal portion 24 of the sheath body 22 to reduce the profile of the distal portion 26 and facilitate navigation in tortuous vasculature. Furthermore, the distal portion 26 may be more flexible than the proximal portion 24. Generally, the proximal portion 24 may be formed from material that is stiffer than the distal portion 26 of the sheath body 22, so that the proximal portion 24 has sufficient pushability to advance through the patient's vascular system, while the distal portion 26 may be formed of a more flexible material so that the distal portion 26 may remain flexible and track more easily over a guidewire to access remote locations in tortuous regions of the vasculature. The sheath body 22 may be composed of suitable polymeric materials, metals and/or alloys, such as polyethylene, stainless steel or other suitable biocompatible materials or combinations thereof. In some instances, the proximal portion 24 may include a reinforcement layer, such a braided layer or coiled layer to enhance the pushability of the sheath body 22. The sheath body 22 may include a transition region between the proximal portion 24 and the distal portion 26.

In general, the vaso-occlusive structure 16 may be inserted into the patient by inserting (e.g., minimally invasively) the vaso-occlusive treatment system 10 into the patient's vasculature to reach the aneurysm site. The delivery catheter 12 is, thus, made as small as possible, and has an extremely narrow inner diameter (i.e., lumen 28) (e.g., between 0.015″ and 0.025″, and preferably between 0.015″ and 0.018″). The vaso-occlusive treatment system 10 may be used in an “over-the-wire” configuration, wherein the delivery catheter 12 is introduced into the patient over a guidewire that has been previously introduced, and the delivery catheter 12 extends over the entire length of the guidewire (not shown). Alternatively, the vaso-occlusive treatment system 10 may be used in a “rapid-exchange” configuration, where a guidewire extends through only a distal portion of the vaso-occlusive treatment system 10 from a guidewire port (not shown). In other alternative embodiments, the vaso-occlusive treatment system 10 may be introduced into the patient after a guidewire had been withdrawn leaving a sheath or access catheter distal portion at the target site for the vaso-occlusive treatment system 10 to navigate through the vasculature of the patient within the sheath or access catheter.

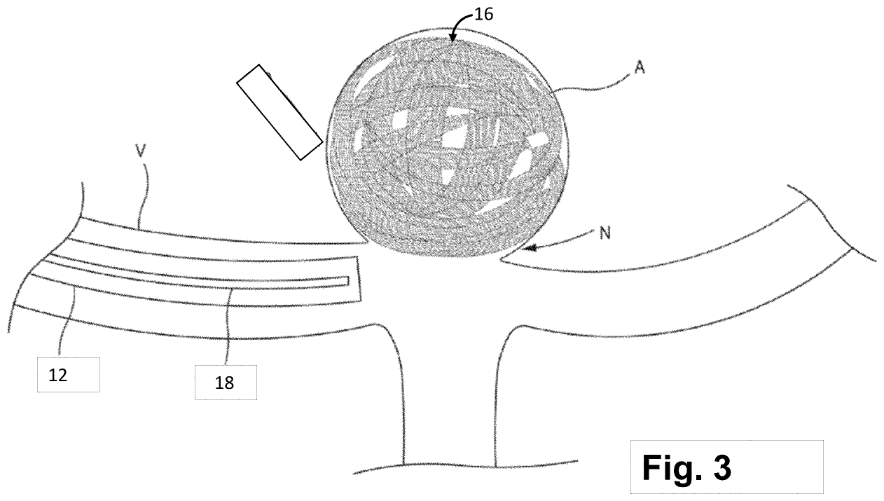

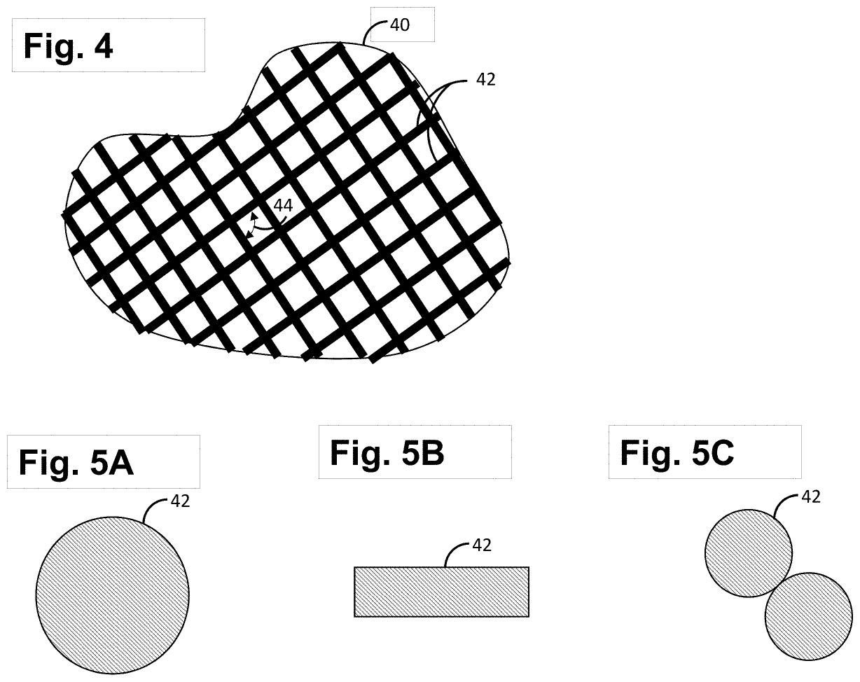

At the aneurysm site, the vaso-occlusive structure 16 may be pushed distally out of the delivery catheter 12 residing in the parent vessel V through the aneurysmal neck N and into an aneurysmal sac A via the pusher member 18, as illustrated in FIG. 3. After being extruded from the delivery catheter 12, the vaso-occlusive structure 16 may self-expand into a pre-set configuration as described below. Once the vaso-occlusive structure 16 is inserted into the aneurysmal sac A, the vaso-occlusive structure 16 may be decoupled from the pusher member 18. A sufficient number of vaso-occlusive devices 16 may be delivered to fill and occlude the aneurysmal sac A. The vaso-occlusive structure 16 may also be removed or withdrawn, and collapsed back into the delivery catheter 12 by proximally withdrawing the vaso-occlusive structure 16 via the pusher member 18.

The pusher member 18 may be a coil, wire, tendon, or the like, having a sufficient columnar strength to permit pushing of the vaso-occlusive structure 16 into the aneurysmal sac. The junction 20 at which the pusher member 18 is coupled to the vaso-occlusive structure 16 may, e.g., take the form of an electrolytically degradable segment for electrolytically decoupling the vaso-occlusive structure 16 from the pusher member 18, although other alternative detachment mechanisms for decoupling the vaso-occlusive structure 16 from the pusher member 18 may include mechanical, thermal, and hydraulic mechanisms.

The pusher member 18 has a proximal portion 32 that extends proximal from the proximal portion 24 of the delivery catheter 12 and a distal portion 34 to which the vaso-occlusive device 14 is attached. The pusher member 18 may be made of a conventional guidewire, torqueable cable tube, or a hypotube. In either case, there are numerous materials that can be used for the pusher member 18 to achieve the desired properties that are commonly associated with medical devices. Some examples can include metals, metal alloys, polymers, metal-polymer composites, and the like, or any other suitable material. For example, the pusher member 18 may include nickel-titanium alloy, stainless steel, a composite of nickel-titanium alloy and stainless steel. In some cases, the pusher member 18 can be made of the same material along its length, or in some embodiments, can include portions or sections made of different materials. In some embodiments, the material used to construct the pusher member 18 is chosen to impart varying flexibility and stiffness characteristics to different portions of the pusher member 18. For example, the proximal region and the distal portion 34 of the pusher member 18 may be formed of different materials, for example materials having different moduli of elasticity, resulting in a difference in flexibility. For example, the proximal portion 32 can be formed of stainless steel, and the distal portion 34 can be formed of a nickel-titanium alloy. However, any suitable material or combination of material may be used for the pusher member 18, as desired.

The vaso-occlusive structure 16 is sized for implantation in an aneurysmal sac A, which can take any geometry or shape in its cross-section. For example, in the illustrated embodiment, the vaso-occlusive structure 16 takes the form of a resilient tubular member having a proximal end 36 and a distal end 38. The distal end 38 of the vaso-occlusive structure 16, in this case, is typically free or loose (allowing maximal expansion) while the proximal end 36 of the vaso-occlusive structure 16 is coupled/attached to the pusher member 18. Thus, the distal end 38 of the vaso-occlusive structure 16 is free-floating. For another example, the vaso-occlusive structure 16 can take the form of flat member where both the proximal and distal ends can be fixed (minimal expansion is allowed). The vaso-occlusive structure 16 has a compact delivery configuration when radially restrained within the delivery catheter 12 and is biased to radially expand outward into a deployed configuration when released from the delivery catheter 12 into the aneurysmal sac. The cross-sectional dimension of the vaso-occlusive structure 16, in its expanded deployed configuration, may, e.g., be greater than 1.5 times, and preferably greater than 2 times, and most preferably, greater than 3 times, the cross-sectional dimension of the vaso-occlusive structure 16, in its compact delivery configuration. The expanded deployed configuration of the vaso-occlusive structure 16 may be pre-set and may be bent, curved, or three-dimensional (e.g., balled-up, looped, etc.), and may include a secondary or tertiary structure.

Significantly, the inventors have discovered that platinum (AuPt) alloy, preferably comprising platinum within the range 25%-40% weight and a Young's modulus of less than 25×10⁶ pounds per square inch (psi), enables the vaso-occlusive structure 16, given a suitable structure, to exhibit the necessary softness (e.g., having a bending stiffness less than 150 mN/mm), desirable length (e.g., greater than 5 cm), compatible with a small diameter delivery catheter (e.g., 0.017″ inner diameter), sufficient radiopacity, sufficient MRI compatibility, and ease of manufacture (e.g., no surface oxide removal requirement). Thus, at least a portion of the vaso-occlusive structure 16 is composed of the AuPt alloy. In addition to an AuPt alloy, the vaso-occlusive structure 16 may be further composed of iridium and/or tungsten to improve its mechanical properties.

In the embodiment illustrated in FIGS. 1 and 2, the entirety of the vaso-occlusive structure 14 comprises a porous mesh portion 40 composed of the AuPt alloy, although as will be discussed in further detail below, only a portion of the vaso-occlusive structure 16 may comprise a mesh portion 40. In the illustrated embodiment, the mesh portion 40 is formed by braiding or weaving wires 42 (e.g., having a wire count in the range of 8-96 wires, typically in the range of 16-32 wires) together, although in alternative embodiments, the mesh portion 40 may be formed as a monolithic structure, e.g., by etching or cutting a pattern from a tube or sheet of stent material, or by cutting or etching a sheet of material according to a desired pattern whereupon the sheet may be rolled or otherwise formed into the desired substantially tubular, bifurcated or other shape.

The mesh portion 40 may have a desired length (e.g., greater than 5 cm, between 5 cm and 45 cm, between 5 cm and 30 cm, etc. Braids may be formed using braiding machines, and may be braided around a mandrel (e.g., a mandrel having a round, oval, flat, other shape depending on the desired final cross-sectional shape of the vaso-occlusive structure 16). Alternatively, wires 42 may be woven into a flat braid and subsequently formed and heat set around a mandrel to a flat braid with a pre-determined shape. After braiding, the mesh portion 40 can be heat set (e.g., at 450° C. to 650° C. for 1 to 60 minutes). The heat set completed braid forms the linear “primary shape” of the mesh portion 40. The heat set completed braid can then be wrapped around a second mandrel (e.g., a three-dimensional mandrel) and heat set for a second time to impart a three-dimensional “secondary shape” or “tertiary shape.”

Each wire 42 may be a monofilament strand, as illustrated in FIGS. 5A and 5B, although in alternative embodiments, each wire 42 may a multi-filament strand, as illustrated in FIG. 5C. Each wire 42 may have any suitable cross-section with any suitable dimension. For example, if the cross-section of each wire 42 is circular (as illustrated in FIG. 5A), the diameter may be in the range of 0.0008″-0.0040″, and if the cross-section of each wire 42 is rectangular (as illustrated in FIG. 5B), the thickness may be greater than 0.0008″, and the width may be less than 0.005″. In another embodiment, each wire 42 may take the form of a twisted wire (as illustrated in FIG. 5C) to enhance the flexibility of the resulting vaso-occlusive structure 16.

Although all of the wires 42 from which the mesh portion 40 is composed may be of identical size and composition, it should be appreciated the wires 42 may have different sizes and composition, as long as at least some of the wires 42 making up the structure of the vaso-occlusive structure 16 are composed of AuPt alloy. Preferably, the mesh portion 40 has an unconstrained braid angle 44 (i.e., the angle between two crossing wires 42) in the range of 20°-130°, more preferably in the range of 20°-60°. In general, a braid angle 44 can be the angle between two crossing wires 42 viewed long the direction of the longitudinal axis. Selecting the braid angle 44 may enhance pushability of the vaso-occlusive structure 16 within the delivery catheter 12 by preventing collapse of the mesh portion 40, which could otherwise result in bunching of the mesh portion 40 in the delivery catheter 12 when pushing and causing jamming of the vaso-occlusive structure 16 within the delivery catheter 12. Ultimately, the number of wires 42 in the mesh portion 40, the braid angle 44, and/or the expanded configuration relative to the collapsed configuration of the mesh portion 40 can be selected to optimally fit the inner diameter of the delivery catheter 12 used.

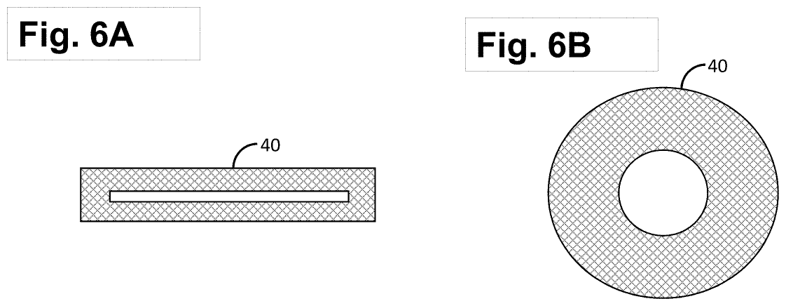

In one embodiment illustrated in FIG. 6A, the mesh portion 40 has an expanded geometry that is flat-shaped (e.g., a ribbon) and may, e.g., have a width in the range of 0.5 mm-5.0 mm, although in an alternative embodiment illustrated in FIG. 6B, the mesh portion 40 may have an expanded geometry that is cylindrical (i.e., has a circular cross-section), and may, e.g., have a diameter in the range of 0.5 mm-5.0 mm. Thus, the mesh portion 40 may be a flat braid or a round braid. Through prototyping and testing, the exact composition of the AuPt alloy, size and number of wires 42 and braid angle used to construct the mesh portion 40 of the vaso-occlusive structure 16, and shape and size of the expanded vaso-occlusive structure 16 can be optimized for superior performance, depending on the requirements of the target application.

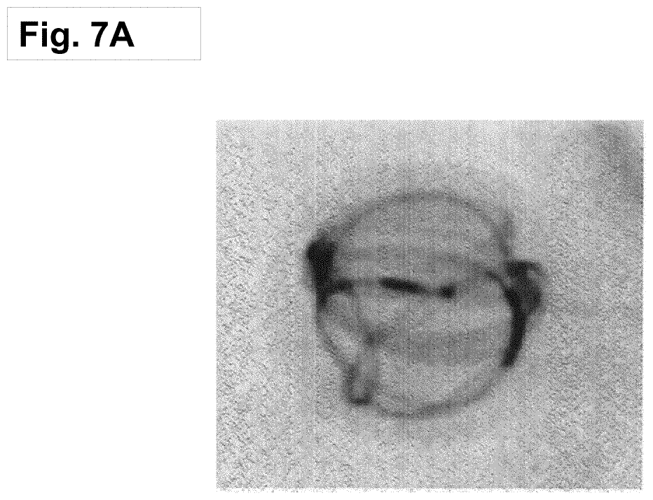

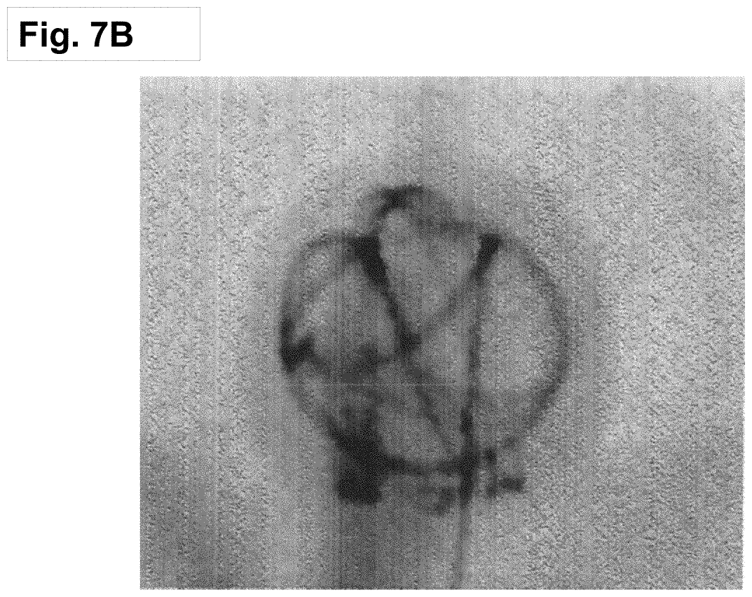

For example, one prototype of a relatively soft and long, but radiopaque, vaso-occlusive device was constructed by braiding twenty-four wires at a braid angle of 32° into a flat braid having a width of 1.25 mm and a length of 25 cm, with each wire being composed of AuPt34 having a Young's modulus of 19 Msi, and having a wire diameter of 0.001″. The vaso-occlusive was deliverable through an Excelsior® SL-10® microcatheter (0.026″ outer diameter and 0.0165″ inner diameter) with a frictional force less than 0.06 lbs, and was demonstrated to have proper shape retention, a good bending stiffness (44.45 mN/mm) and good radiopacity at x-ray energy of 82 KVp, as illustrated in FIG. 7A.

As another example, another prototype of a relatively soft and long, but radiopaque, vaso-occlusive device was constructed by braiding twenty-four wires at a braid angle of 32° into a flat braid having a width of 1.25 mm and a length of 25 cm, with each wire being composed of AuPt29 having a Young's modulus of 17 Msi, and having a wire diameter of 0.00115″. The vaso-occlusive was deliverable through an Excelsior® SL-10® microcatheter (0.026″ outer diameter and 0.0165″ inner diameter) with a frictional force less than 0.06 lbs, and was demonstrated to have proper shape retention, a good bending stiffness (67.33 mN/mm) and good radiopacity at X-ray energy of 82 KVp. It is noted that, although this vaso-occlusive device is not as soft as the vaso-occlusive device discussed immediately above (67.33 mN/mm versus 44.45 mN/mm), this vaso-occlusive device has a better radiopacity, as illustrated in FIG. 7B.

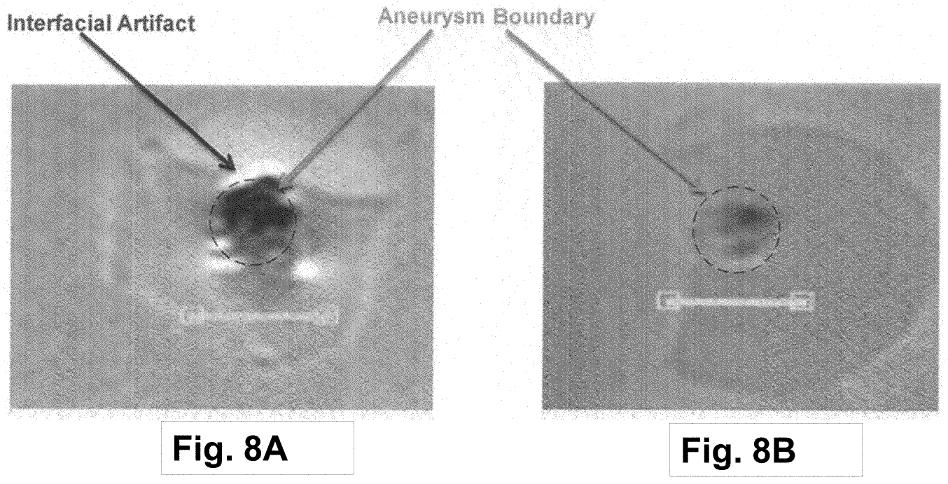

As still another example, the MR compatibility properties of a prototype of vaso-occlusive device in the form of a helically wound coil composed of AuPt29 were compared to a helically wound coil composed of conventional Pt/8W. A 6 mm aneurysm was filled with the vaso-occlusive coils composed of AuPt29 at a 35% packing density and the site was imaged with an MRI at 3T (see FIG. 8B), while the same 6 mm aneurysm was filled with the vaso-occlusive coils composed of conventional Pt/8W at a 35% packing density and the site was imaged with an MRI at 3T (see FIG. 8A). As can be appreciated that, the MRI image of the conventional Pt/8W vaso-occlusive coils has artifacts, such as an interfacial artifact, whereas the MRI image of the novel AuPt29 vaso-occlusive coils advantageously lacks such interfacial artifact.

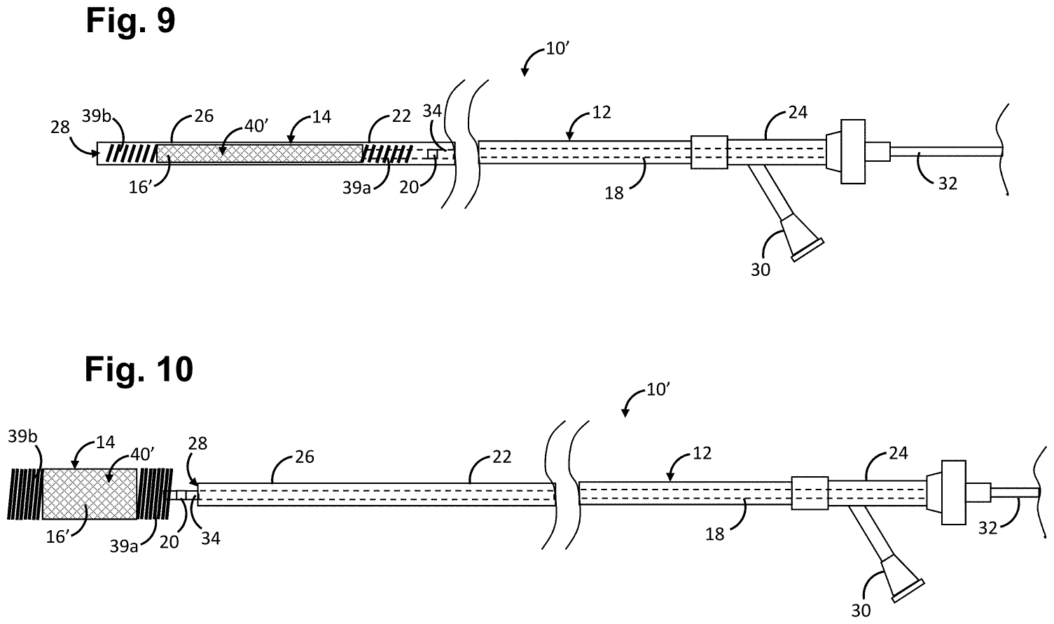

As briefly discussed above, only a portion of the vaso-occlusive structure 16 may comprise a mesh portion 40. For example, as illustrated in FIGS. 9 and 10, another embodiment of a vaso-occlusive treatment system 10′ constructed in accordance with the present inventions will now be described. The vaso-occlusive treatment system 10′ is similar to the vaso-occlusive treatment system 10, with the exception that the vaso-occlusive structure 16′ comprises a central mesh portion 40′ and two helically wound coil portions 39a, 39b disposed at opposite ends of the central mesh portion 40′. The central mesh portion 40′ can be constructed in the same manner as the mesh portion 40 described with respect to FIGS. 1 and 2. Preferably, the coil portions 39a, 39b are composed of a AuPt alloy. Notably, the coil portions 39a, 39b provide additional non-traumatic characteristics to the vaso-occlusive structure 16′.

Although the vaso-occlusive structures 16, 16′ respectively illustrated in FIGS. 1-2 and 9-10 have been described as having a single layer of braid, it should be appreciated the a vaso-occlusive structure may comprise multiple layers of braid (i.e, braid over braid construction), or may even comprise one braid layer (e.g., an outer layer of braid) and a coil layer (e.g., an inner coil) (i.e., a braid over coil construction). In either event, all layers of the vaso-occlusive structure are preferably composed of a AuPt alloy.

Although particular embodiments of the disclosed inventions have been shown and described herein, it will be understood by those skilled in the art that they are not intended to limit the present inventions, and it will be obvious to those skilled in the art that various changes and modifications may be made (e.g., the dimensions of various parts) without departing from the scope of the disclosed inventions, which is to be defined only by the following claims and their equivalents. The specification and drawings are, accordingly, to be regarded in an illustrative rather than restrictive sense. The various embodiments of the disclosed inventions shown and described herein are intended to cover alternatives, modifications, and equivalents of the disclosed inventions, which may be included within the scope of the appended claims.

What is claimed is:

1. A vaso-occlusive device, comprising:
an elongate vaso-occlusive structure configured for implantation in an aneurysm sac, the vaso-occlusive structure having a delivery configuration when restrained within a delivery catheter and having a deployed configuration when released from the delivery catheter into the aneurysmal sac, at least a portion of the vaso-occlusive structure being composed of a gold-platinum (AuPt) alloy.

2. The vaso-occlusive device of claim 1, wherein the AuPt alloy comprises platinum within the range 25%-40% by weight.

3. The vaso-occlusive device of claim 1, wherein the AuPt alloy has a Young's modulus less than 25×10⁶ pounds per square inch (psi).

4. The vaso-occlusive device of claim 1, wherein the vaso-occlusive structure comprises a mesh portion composed of the AuPt alloy.

5. The vaso-occlusive device of claim 4, wherein the mesh portion is a braided portion.

6. The vaso-occlusive device of claim 4, wherein the entirety of the vaso-occlusive structure comprises the mesh portion.

7. The vaso-occlusive device of claim 4, wherein the vaso-occlusive structure further comprises two helically wound coil portions disposed at opposite ends of the mesh portion.

8. The vaso-occlusive device of claim 7, wherein each of the two helically wound coil portions is composed of the AuPt alloy.

9. The vaso-occlusive device of claim 4, wherein the mesh portion comprises at least one wire, each having a minimum cross-sectional dimension in the range of 0.0008″-0.004″.

10. The vaso-occlusive device of claim 4, wherein the mesh portion comprises at least one twisted strand.

11. The vaso-occlusive device of claim 4, wherein the mesh portion has a wire count in the range of 8-96 wires.

12. The vaso-occlusive device of claim 4, wherein the mesh portion has a wire count in the range of 16-32 wires.

13. The vaso-occlusive device of claim 4, wherein the mesh portion has an unconstrained braid angle in the range of 20°-60°.

14. The vaso-occlusive device of claim 1, wherein the mesh portion has an expanded geometry having a circular cross-section.

15. The vaso-occlusive device of claim 1, wherein the mesh portion has an expanded geometry having a rectangular cross-section.

16. The vaso-occlusive device of claim 15, wherein the rectangular cross-section has a width in the range of 1.0 mm-2.0 mm.

17. The vaso-occlusive device of claim 15, wherein the mesh portion has a bending stiffness less than 150 mN/mm.

18. The vaso-occlusive device of claim 1, wherein the vaso-occlusive structure is further composed of one or both of Iridium and Tungsten.

19. A vaso-occlusive assembly, comprising:
the vaso-occlusive device of claim 1; and
a pusher member to which the vaso-occlusive device is detachably coupled.

20. A vaso-occlusive treatment system, comprising:
a vaso-occlusive assembly of claim 19; and
the delivery catheter in which the vaso-occlusive assembly is disposed.
